(19) United States
(12) Patent Application Publication
(10) Pub. No.: US 2023/0093312 A1
(43) Pub. Date: Mar. 23, 2023

(54) METHOD AND SYSTEM FOR DETERMINING CONDITION-SPECIFIC HEALTH, COGNITIVE AND/OR BEHAVIOR ANOMALIES BASED ON MONITORED DAILY ACTIVITY OF A USER

(71) Applicant: M.You Cognitive Technologies LTD, Tel Aviv - Yafo (IL)

(72) Inventors: Itay BARUCHI, Tel Aviv - Yafo (IL); Ruth POLIAKINE, Tel Aviv - Yafo (IL)

(73) Assignee: M.You Cognitive Technologies LTD, Tel Aviv - Yafo (IL)

(21) Appl. No.: 17/801,844

(22) PCT Filed: Feb. 21, 2021
(86) PCT No.: PCT/IL2021/050199

§ 371 Date: Aug. 24, 2022

Related U.S. Application Data
(60) Provisional application No. 62/982,152, filed on Feb. 27, 2020.

(51) Int. Cl. G16H 50/20; G16H 50/50; G16H 40/67

(52) U.S. Cl. CPC G16H 50/20; G16H 50/50; G16H 40/67

(57) ABSTRACT

A method of determining a condition of a user, the method may include: monitoring one or more activities performed by a user during a predetermined monitoring time period; determining one or more activity complexity datasets each for one of the one or more monitored activities; determining a user complexity model based on at least one of the one or more activity complexity datasets; determining a distance vector between the user complexity model and a reference user complexity model; and determining a condition of the user based on the distance vector and a reference distance-condition model.

### FIELD OF THE INVENTION

The present invention relates to the field of determining condition-specific health, cognitive and/or behavior anomalies of a user and, more particularly, to determining condition-specific health, cognitive and/or behavior anomalies based on monitored daily activity of the user.

### BACKGROUND OF THE INVENTION

Daily activity of a user may be affected by user's health, cognitive and/or behavioral condition.

### SUMMARY OF THE INVENTION

One aspect of the present invention may provide a method of determining a condition of a user, the method may include: monitoring one or more activities performed by a user during a predetermined monitoring time period; determining one or more activity complexity datasets each for one of the one or more monitored activities; determining a user complexity model based on at least one of the one or more activity complexity datasets; determining a distance vector between the user complexity model and a reference user complexity model; and determining a condition of the user based on the distance vector and a reference distance-condition model.

Some embodiments may include monitoring the one or more activities by obtaining one or more time series of data points for each of the one or more monitored activities during the predetermined monitoring time period from one or more sensors wearable by the user; and determining the one or more activity complexity datasets, each for one of the one or more monitored activities, based on at least one of the one or more time series obtained for the respective monitored activity.

Some embodiments may include determining at least one of: whether the user has an anomaly and a probability that the user has the anomaly, based on the distance vector; and determining the condition of the user when it has been determined that the user has the anomaly or when the probability thereof is above a predetermined probability threshold.

Some embodiments may include determining whether the user has the anomaly based on at least one of: a norm of the distance vector and a predetermined norm threshold; one or more distance values in one or more dimensions of the distance vector and one or more predetermined distance thresholds; and a pre-trained machine learning model.

Some embodiments may include determining the probability that the user has the anomaly based on at least one of: a reference anomaly probability logistic model; and a pre-trained machine learning model.

Some embodiments may include determining a condition of the user based on at least one of: a norm of the distance vector; and one or more distance values in one or more dimensions of the distance vector.

Some embodiments may include that determining the condition of the user comprises determining one of: a specific condition of the user; or a group of conditions from which the user may suffer.

Some embodiments may include tracking the condition of the user as the time progresses; and determining a trend of the condition of the user based on the tracking thereof.

Some embodiments may include predetermining the reference user complexity model by monitoring the one or more activities during a predetermined reference time period prior to the predetermined monitoring time period.

Another aspect of the present invention may provide a system for determining a condition of a user, the system may include: an activity complexity dataset determination module configured to:

obtain one or more time series of data points for each of one or more monitored activities during a predetermined monitoring time period, and determine the one or more activity complexity datasets, each for one of the one or more monitored activities, based on at least one of the one or more time series obtained for the respective monitored activity; a user complexity model determination module configured to determine a user complexity model based on at least one of the one or more activity complexity datasets; a distance vector determination module configured to determine a distance vector between the user complexity model and a reference user complexity model; and a user condition determination module configured to determine a condition of the user based on the distance vector and a reference distance-condition model.

In some embodiments, the system may include an anomaly determination model that is configured to determine at least one of: whether the user has an anomaly and a probability that the user has the anomaly, based on the distance vector, wherein the user condition determination model is configured to determine the condition of the user when it has been determined that the user has the anomaly or when the probability thereof is above a predetermined probability threshold.

In some embodiments, the anomaly determination model is further configured to determine whether the user has the anomaly based on at least one of: a norm of the distance vector and a predetermined norm threshold; one or more distance values in one or more dimensions of the distance vector and one or more predetermined distance thresholds; and a pre-trained machine learning model.

In some embodiments, the anomaly determination model is further configured to determine the probability that the user has the anomaly based on at least one of: a reference anomaly probability logistic model; and a pre-trained machine learning model.

In some embodiments, the user condition determination module is configured to determine a condition of the user based on at least one of: a norm of the distance vector; and one or more distance values in one or more dimensions of the distance vector.

In some embodiments, the user condition determination module is configured to determine one of: a specific condition of the user; or a group of conditions from which the user may suffer.

In some embodiments, the system may include a user condition tracking module configured to: track the condition of the user as the time progresses; and determine a trend of the condition of the user based on the tracking thereof.

In some embodiments, the system may include a reference user complexity model determination module configured to predetermine the reference user complexity model by monitoring the one or more activities during a predetermined reference time period prior to the predetermined monitoring time period.

These additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIG. 1 is a schematic block diagram of a system for determining a condition of a user, according to some embodiments of the invention;

FIG. 2 is a schematic illustration of a process of determining an activity complexity dataset including a single activity complexity data value, according to some embodiments of the invention;

FIG. 3 is a schematic illustration of a process of determining an activity complexity dataset including a one-dimensional (1D) activity complexity data values probability distribution, according to some embodiments of the invention;

FIG. 4 is a schematic illustration of a first example of pre-pre-processing and of a process of determining an activity complexity dataset including a multi-dimensional activity complexity data values probability distribution, according to some embodiments of the invention;

FIG. 5 is a schematic illustration of a second example of pre-pre-processing and of a process of determining an activity complexity dataset, according to some embodiments of the invention;

FIG. 6 is a schematic illustration of a user complexity model including a multi-dimensional user complexity data values distribution, according to some embodiments of the invention;

FIG. 7 is a schematic illustration of a user complexity model, a reference user complexity model and of a distance vector therebetween, according to some embodiments of the invention;

FIG. 8 is a schematic illustration of a reference anomaly probability logistic model, according to some embodiments of the invention;

FIG. 9 is a schematic illustration of a reference distance-condition model, according to some embodiments of the invention;

FIG. 10 is a schematic illustration of a trend of distance vectors, according to some embodiments of the invention; and

FIG. 11 is a flowchart of a method of determining a condition of a user, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

Unless specifically stated otherwise, as apparent from the following discussions, it is appreciated that, throughout the specification, discussions utilizing terms such as “processing”, “computing”, “calculating”, “determining”, “enhancing” or the like refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Any of the disclosed modules or units can be at least partially implemented by a computer processor.

Some aspects of the present invention may provide a method and a system for determining a condition of a user. The condition may, for example, be at least one of: health, cognitive and/or behavioral condition.

Some embodiments may include monitoring one or more activities performed by a user during a predetermined monitoring time period. The one or more monitored activities may include any daily activity performed by the user. Such daily activities may, for example, include voice activity, walking, driving, running, etc. In some embodiments, more than one activity may be monitored.

Some embodiments may include determining one or more activity complexity datasets each for one of the one or more monitored activities. The activity complexity dataset determined for a particular activity may, for example, present a highly compressed description of regularities of this particular activity. In other words, the activity complexity dataset determined for the particular activity may represent a length of description of regularities of this particular activity.

For example, one of the monitored activities may be a voice activity of the user. A voice activity complexity dataset may, for example, represent an ability of the user to dynamically change voice patterns by changing the voice pitch, power and space between syllables, words and/or to convey addition information (e.g., in addition to the information conveyed by the words themselves) such as, for example, pausing before a word for emphasis, increasing an intensity of the voice for certain words or changing the voice pitch for certain sentences.

Some embodiments may include determining a user complexity model based on at least one of the one or more activity complexity datasets. In some embodiments, the user complexity model may be determined by combining different activity complexity datasets determined for different monitored activities.

Some embodiments may include determining a distance vector between the user complexity model and a reference user complexity model. The reference user complexity model may be predetermined by, for example, monitoring the one or more activities of the user during, for example, 2-4 weeks prior to the monitoring time period. The reference user complexity model may, for example, represent a baseline model for the user complexity.

A change in a complexity of one or more activities performed by the user may be indicative of, for example, a change in health, cognitive and/or behavioral condition of the user. Referring to the example of voice activity, a cognitive change of the user may affect the ability of the user to dynamically change its voice patterns and/or generate a plurality of voice patterns which may be reflected in the voice activity complexity. For example, a reduction in the cognitive ability of the user may reduce the ability of the user to dynamically change its voice patterns which may result in reduction of its voice activity complexity. Accordingly, the distance vector determined based on the user complexity model and the reference user complexity model may be indicative of a change in health, cognitive and/or behavioral condition of the user.

Various embodiments may include determining whether the user has an anomaly or a probability that the user has the anomaly based on the distance vector. For example, one or more predetermined distance thresholds, a reference anomaly probability logistic model and/or a trained machine learning method may be applied to the distance vector or to one or more distance values in one or more dimensions of the distance vector to determine whether the user has the anomaly or the probability thereof.

Some embodiments may include determining, for example once it has been determined that the user has the anomaly or that the probability thereof is above a predetermined probability threshold, a condition of the user based on the distance vector and a reference distance-condition model. The condition of the user may, for example, be health, cognitive and/or behavioral condition. In various embodiments, the condition of the user may relate to a specific health, cognitive and/or behavior condition or to a group of health, cognitive and/or behavior conditions.

Some embodiments may include tracking the condition of the user as the time progresses and determining a trend of the condition of the user based on the tracking thereof.

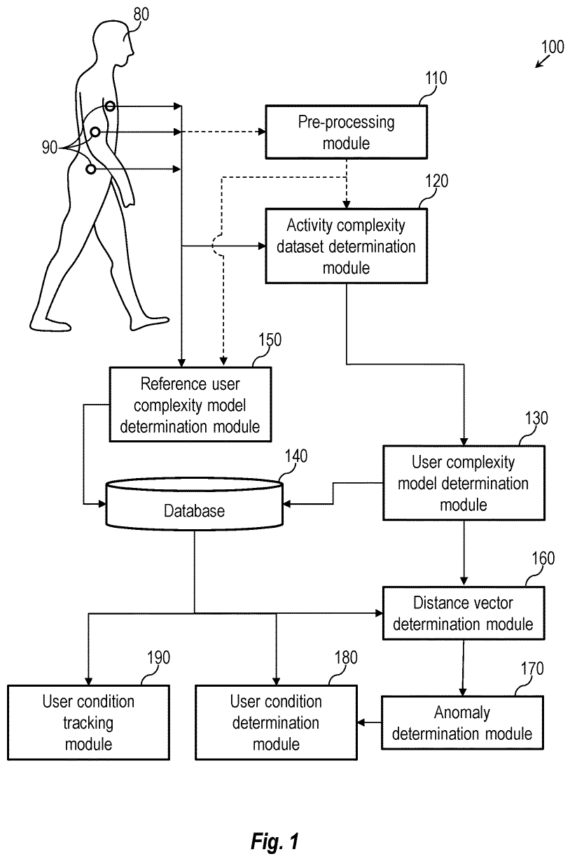

Reference is now made to FIG. 1, which is a schematic block diagram of a system 100 for determining a condition of a user 80, according to some embodiments of the invention.

According to some embodiments, system 100 may include a pre-processing module 110, an activity complexity dataset determination module 120, a user complexity model determination module 130, a database 140, a reference user complexity model determination module 150, a distance vector determination module 160, an anomaly determination module 170, a user condition determination module 180 and a user condition tracking module 190.

System 100 (or at last some modules thereof) may be in communication with one or more sensors 90. Sensor(s) 90 may be wearable by user 80. Sensor(s) 90 may, for example, include one or more accelerometer(s), gyro(s), magnetometer(s), microphone(s), GPS(s), etc. For example, sensor(s) 90 may be sensor(s) embedded in a mobile phone of user 80. In this case, system 100 (or at least some modules thereof) may interface with the mobile phone of user 90 to obtain data from the sensor(s) thereof.

In some embodiments, sensor(s) 90 may be part of system 100. For example, system 100 may include a hardware including sensor(s) 90, wherein the hardware may be wearable by user 80.

Sensor(s) 90 may be controlled by system 100 to monitor one or more activities of user 90 (e.g., daily activities such as voice activity, walking, driving, etc.). Sensor(s) 90 may monitor the one or more activities by obtaining one or more time series of data points for each of the one or more monitored activities during a predetermined monitoring time period. The predetermined monitoring time period may, for example, range between 1-2 weeks. For example, once system 100 detects that the user performs one of the one or more activities during the monitoring time period, sensor(s) 90 may be activated to obtain the time series.

In a first example, sensor(s) 90 may monitor two activities: a first activity using a first sensor of sensors 90 (e.g., walking using an accelerometer sensor) and a second activity using a second sensor of sensors 90 (e.g., voice activity using microphone sensor). The first sensor may obtain multiple time series of data points for the first activity, and the second sensor may obtain multiple time series of data points for the second activity during the predetermined monitoring time period.

In some embodiments, different sensors of sensors 90 may monitor different activities. For example, an accelerometer sensor may monitor walking of user 80, and a microphone sensor may monitor voice activity of user 80 (e.g., as described in the first example above). In some other embodiments, at least some of sensors 90 may monitor more than one activity. For example, an accelerometer sensor may monitor both walking and voice activity of user 80. In some embodiments, one activity may be monitored by more than one sensor of sensors 90. For example, walking may be monitored by an accelerometer and by a gyro sensor.

In some embodiments, activity complexity dataset determination module 120 may determine one or more activity complexity datasets each for one of the one or more monitored activities based on at least one of the one or more time series obtained for the respective monitored activity.

Referring to the first example, a first activity complexity dataset may be determined for the first activity based on at least one of the time series obtained by the first sensor for the first activity, and a second activity complexity dataset may be determined based on at least one of the time series obtained by the second sensor for the second activity.

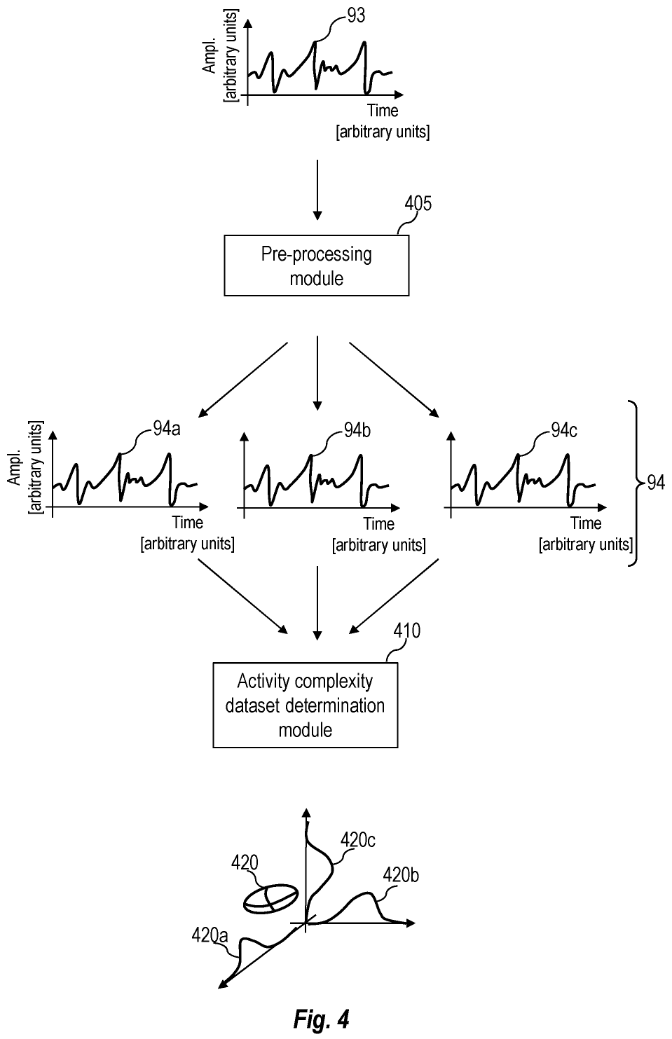

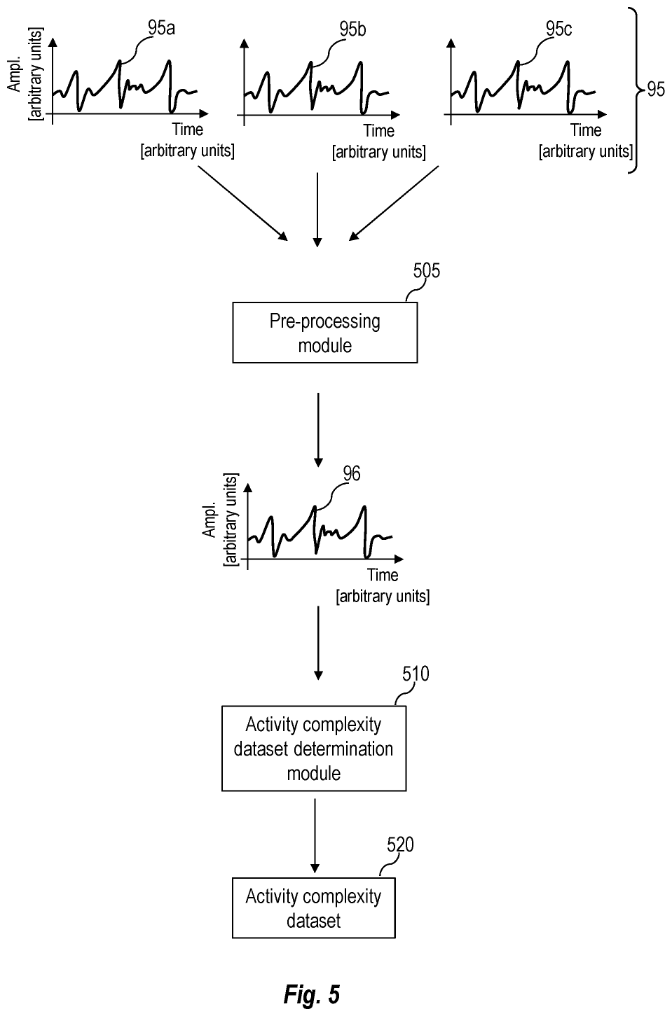

In some embodiments, one or more of the time series obtained by sensor(s) 90 may be first inputted to pre-processing module 110 for pre-processing to yield one or more pre-processed time series. The one or more pre-processed time series may be then transmitted to activity complexity dataset determination module 120 that may determine at least a portion of at least one activity complexity dataset based on the one or more pre-processed time series. It is noted that the pre-processing may be optional, as indicated by dashed arrows in FIG. 1. Some examples of pre-processing are described below with respect to FIGS. 4 and 5. However, other pre-processing may be also performed. For example, time series that includes voice data may be preprocessed to obtain a signal intensity and pitch of each voice segment. In another example, time series that includes accelerometer data obtained from three axes may be preprocessed using dimension reduction (e.g., such as principal component analysis or independent component analysis) to obtain the main moving direction while walking or driving. In another example, for a time series that include accelerometer may can be preprocessed into a time-frequency image using, e.g., short term Fourier transform. In another example, time series that include voice data may be preprocessed into silent and voice segments to obtain a binary time series.

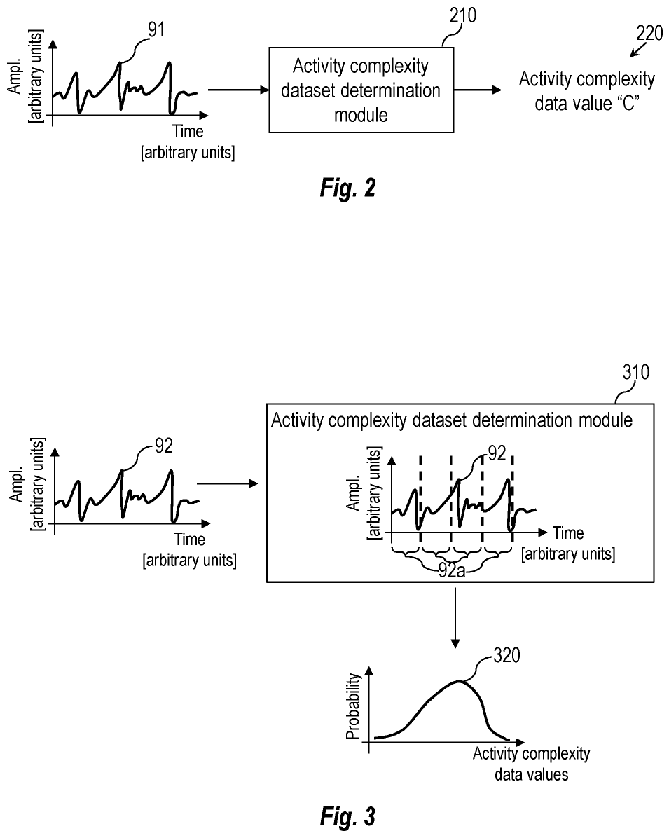

In some embodiments, each of the one or more activity complexity datasets may include at least one activity complexity data value. For example, each of the one or more activity complexity datasets may include a single activity complexity data value (e.g., as described below with respect to FIG. 2) or a dataset of activity complexity data values. For example, the dataset of the activity complexity data values may include a one-dimensional (1D) activity complexity data values probability distribution (e.g., as described below with respect to FIGS. 3), a two-dimensional (2D) activity complexity data values probability distribution, a three-dimensional (3D) activity complexity data values probability distribution, or a multi-dimensional activity complexity data values probability distribution (e.g., as described below with respect to FIGS. 3).

In some embodiments, user complexity determination module 130 may determine a user complexity model for user 80 based on at least one of the one or more activity complexity datasets. In some embodiments, the user complexity model may be stored in database 140.

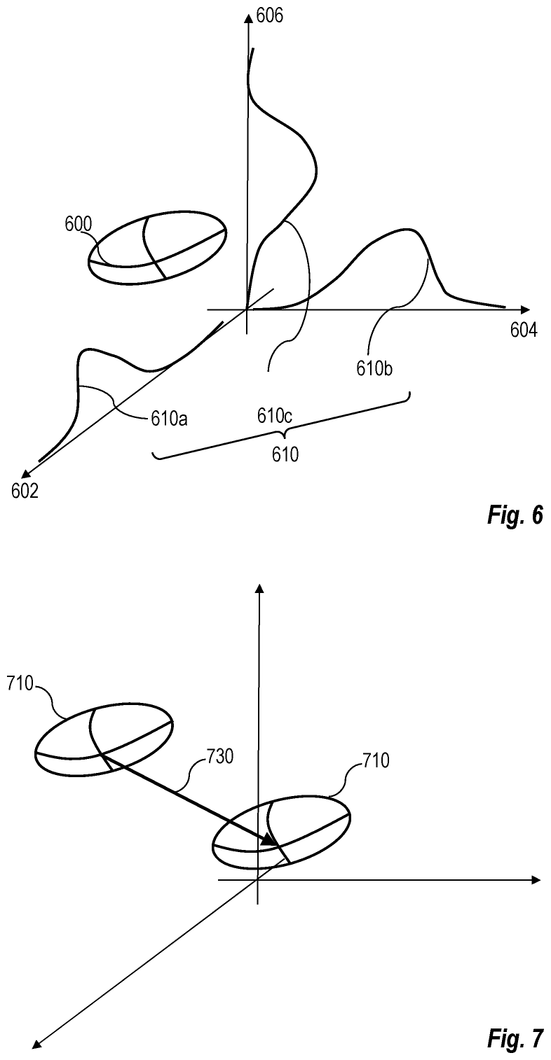

The user complexity model may be determined by, for example, combining different activity complexity datasets determined for different monitored activities. The user complexity model may include at least one user complexity data value or a dataset of user complexity data values (e.g., 1D, 2D, 3D or multi-dimensional datasets). One example of the user complexity determination module is described below with respect to FIG. 6.

Referring to the first example, each of the first activity complexity dataset and the second activity complexity dataset may include 1D activity complexity data values probability distribution and a corresponding first user complexity model may thus include 2D user complexity data values probability distribution.

In some embodiments, user complexity determination module 130 may determine a set of two or more user complexity models for the same user 80 during two or more different (e.g., subsequent) monitoring time periods. At least some of the user complexity models of the set may be stored in database 140.

In some embodiments, distance vector determination module 160 may determine a distance vector between the user complexity model and a reference user complexity model. The reference user complexity model may represent a baseline model for the user complexity. The reference user complexity model may be determined by reference user complexity model determination module 150 based on reference time series, or optionally pre-processed reference time series, obtained by sensor(s) 90 for the one or more monitored activities during a predetermined reference time period. For example, the reference time period may range 2-4 weeks prior to, for example, the monitoring time period. The reference user complexity model may be stored in a database 140.

The distance vector between the user complexity model and the reference user complexity model may be determined using a variety of metrics, such as, for example, Euclidean, correlation, Chebyshev distance, subtraction, etc. The distance vector may also take into account the variance between the user complexity model and the reference user complexity model using methods such as, for example, effect size or Mahalanobis distance.

The distance vector between the user complexity model and the reference user complexity model may have at least one distance vector dimension. The number of dimensions of the distance vector may correspond to, for example, the number of dimensions of the user complexity model. Referring to the first example, the distance vector may have two dimensions as the number of dimensions of the first user complexity model.

One example of the reference user complexity model and of the distance vector are described below with respect to FIG. 7.

In the case when two or more user complexity models are determined for the same user 80 during two or more different monitoring time periods, distance vector determination module 160 may determine a set of two or more distance vectors each corresponding to one of the two or more user complexity models. In some embodiments, at least some of the distance vectors of the set may be stored in database 140.

In some embodiments, anomaly detection module 170 may determine whether user 80 has an anomaly based on the distance vector and one or more predetermined thresholds. The anomaly may be, for example, health, cognitive and/or behavioral anomaly. For example, a norm of the distance vector that is above a predetermined norm threshold and/or one or more distance values in one or more dimensions of the distance vector that is above a predetermined distance threshold may be indicative of, for example, an anomaly of user 80.

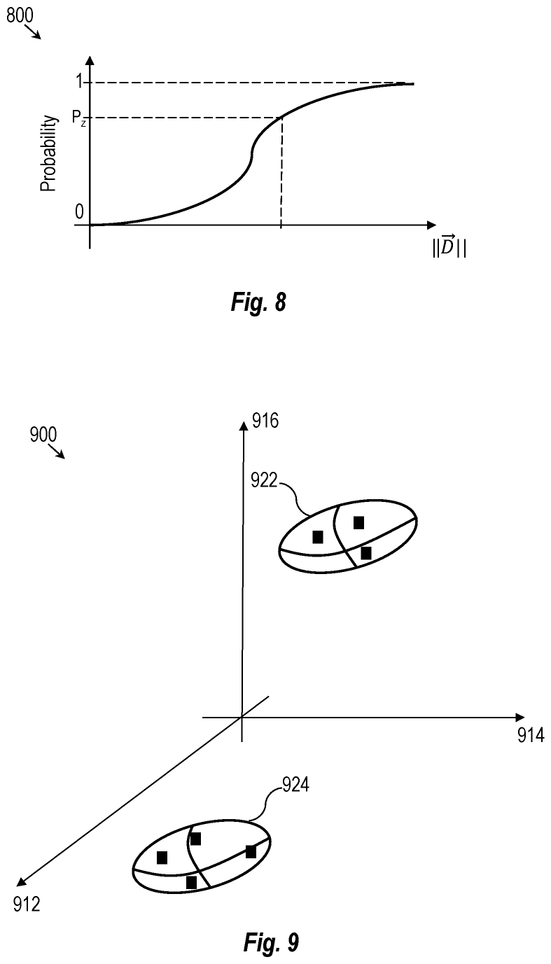

In some embodiments, anomaly detection module 170 may determine a probability that user 80 has an anomaly based on the distance vector and a reference anomaly probability logistic model. For example, a single probability value may be determined based on the norm of the distance vector and/or one or more probability values may be determined based on one or more distance values in one or more dimensions of the distance vector. The reference anomaly probability logistic model may be determined based on, for example, training data collected from users who have been diagnosed with some level of, for example, health, cognitive and/or behavioral decline or with a deterioration of health, cognitive and/or behavioral condition that is known to affect cognitive ability. One example of the reference anomaly probability logistic model is described below with respect to FIG. 8.

In various embodiments, anomaly detection module 170 may determine whether user 80 has an anomaly and/or determine a probability that user 80 has the anomaly based on the distance vector and using a pre-trained machine learning model. For example, the norm of the distance vector and/or one or more of distance values in one or more dimensions of the distance vector may be inputted into the pre-trained machine learning model that may output whether user 80 has the anomaly and/or the probability that the user has the anomaly.

In some embodiments, as long as no anomaly is detected, the operations of determining the user complexity model (e.g., by user complexity model determination module 130), determining the distance vector (e.g., by distance vector determination module 160) and analyzing the distance vector (e.g., by anomaly determination module 170) may be repeated, for example, every few weeks (e.g., 1-2 weeks), every few days (e.g., 1-2 days) or every day, depending on the predetermined monitoring time period.

In various embodiments, for example when it has been determined that the user has the anomaly or the probability that the user has the anomaly is above a predetermined probability threshold, user condition determination model 180 may determine a condition of the user based on the distance vector and a reference distance-condition model. The condition of the user may, for example, be health, cognitive and/or behavioral condition. In various embodiments, the condition of the user may relate to a specific health, cognitive and/or behavior condition (e.g., chronic obstructive pulmonary disease, urinary tract infection, sepsis in diabetes patients, etc.) or to a group of health, cognitive and/or behavior conditions.

In some embodiments, the condition of the user may be determined based on the norm of the distance vector. For example, the norm of the distance vector that is within a first predetermined range may indicate that user 80 has a first condition, and the norm of the distance vector that is within a second predetermined range may indicate that user 80 has a second condition.

In some embodiments, the condition of the user may be determined based on one or more distance values in one or more dimensions of the distance vector. For example, user 80 that suffers from chronic obstructive pulmonary disease and developing a decrease abstraction may show an anomaly in an ability to talk and an ability to walk, which may be manifested in large distance values of the distance vector that relate to voice activity complexity dimension and walking complexity dimension as compared to distance values that relate to other complexity dimensions related to other activities, such as driving. In another example, some types of Aphasia may cause an anomaly in voice activity complexity dimension but not in walking complexity dimension.

The reference distance-condition model may, for example, include a variety of multiclass classification methods such as, for example, a support vector machine (SVM), random forest, Bayesian method, etc. The reference distance-condition model may be determined based on, for example, training data collected from users who have been diagnosed with specific conditions. One example of the reference distance-condition model is described below with respect to FIG. 9.

In some embodiments, user condition tracking module 190 may track the condition of user 80 as the time progresses. User condition tracking module 190 may determine a trend of the condition of user 90 based on the tracking thereof.

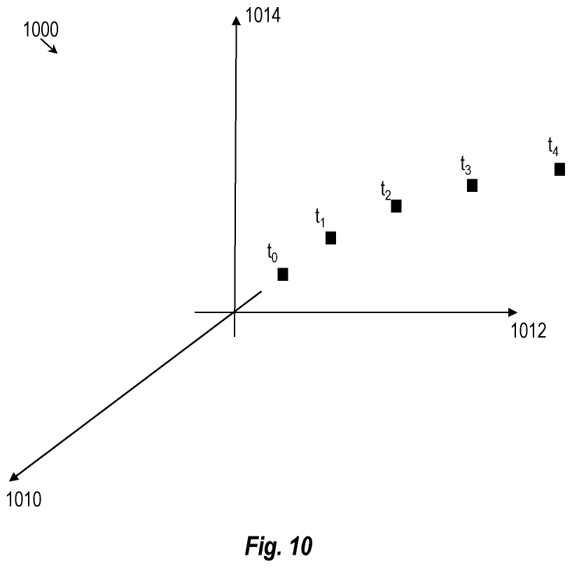

For example, user condition tracking module 190 may track at least some of the two or more distance vectors determined for two or more different user complexity models determined for the same user 80 during two or more different monitoring time periods. One example of tracking of distance vectors in time is described below with respect to FIG. 10. The tracking may be based on, for example, norms of the distance vectors and/or based on distance values in corresponding dimensions of the distance vectors. User condition tracking module 190 may determine a trend of the two or more distance vectors as the time progresses based on the tracking thereof. The trend may be indicative of, for example, a trend of the condition of user 80 in time. For example, at time to, user 80 may not show any sign associated with health, cognitive and/or behavioral decline, but, as the time progresses, a distance between the user complexity model and the reference user complexity model may, for example, increase, which may, for example, indicate the health, cognitive and/or behavioral decline thereof. Tracking and determining the trend thereof may, for example, enable an early detection of the condition of user 80 or a change thereof.

As would be apparent to those of ordinary skill in the art, each module and/or database of system 100 can be implemented on its own computing device, a single computing device, or a combination of computing devices. The communication between the modules and/or database of system 100 can be wired and/or wireless.

Reference is now made to FIG. 2, which is a schematic illustration of a process of determining an activity complexity dataset 220 including a single activity complexity data value, according to some embodiments of the invention.

FIG. 2 depicts, for example, a time series 91 obtained for an activity performed by a user during a predetermined monitoring time period. Time series 91 may be obtained by, for example, one or more sensors, such as sensor(s) 90 described above with respect to FIG. 1. Time series 91 may be inputted into an activity complexity dataset determination module 210 (e.g., such as activity complexity dataset determination module 120 described above with respect to FIG. 1). Activity complexity dataset determination module 210 may determine an activity complexity dataset 220 (e.g., as described above with respect to FIG. 1). Activity complexity dataset 220 may include a single activity complexity data value “C”.

Reference is now made to FIG. 3, which is a schematic illustration of a process of determining an activity complexity dataset 320 including a one-dimensional (1D) activity complexity data values probability distribution, according to some embodiments of the invention.

FIG. 3 depicts, for example, a time series 92 obtained for an activity performed by a user during a predetermined monitoring time period. Time series 92 may be obtained by, for example, one or more sensors, such as sensor(s) 90 described above with respect to FIG. 1. Time series 92 may be inputted into an activity complexity dataset determination module 310 (e.g., such as activity complexity dataset determination module 120 described above with respect to FIG. 1).

In some embodiments, activity complexity dataset determination module 310 may divide time series 92 into multiple subsets 92a of data points. For example, time series 92 may be divided into multiple subsets 92a of data points such that each of subsets 92a may correspond to a predetermined time window during which the data points of the respective subset have been obtained. The time windows may range between few seconds, minutes, hours or days, with or without overlaps between subsequent time windows.

Activity complexity dataset determination module 310 may determine an activity complexity dataset 320. Activity complexity dataset 320 may include multiple activity complexity data values each determined based on one of multiple subsets 92a. The multiple activity complexity data values may be presented as a 1D activity complexity data values probability distribution (e.g., such as a Gaussian distribution in case of scalar activity complexity data values or a multivariate Gaussian distribution in case of an n-dimensional activity complexity data vector).

Reference is now made to FIG. 4, which is a schematic illustration of a first example of pre-pre-processing and of a process of determining an activity complexity dataset 420 including a multi-dimensional activity complexity data values probability distribution, according to some embodiments of the invention.

FIG. 4 depicts, for example, a time series 93 obtained for an activity performed by a user during a predetermined monitoring time period. Time series 93 may be obtained by, for example, one or more sensors, such as sensor(s) 90 described above with respect to FIG. 1.

In some embodiments, time series 93 may be inputted into a pre-processing module 405 (e.g., such as pre-processing module 110 described above with respect to FIG. 1). Pre-processing module 405 may determine two or more time-subseries 94 based time series 93.

For example, FIG. 4 depicts an example in which three time-subseries 94 are determined based on time series 93. For example, if time series 93 represents a recorded speech of a user, a first time-subseries 94a may represent a voice pitch value as function of time, a second time-subseries 94b may represent a voice intensity as function of time, and a third time-subseries 94c may represent a voice segment duration as function of time.

In some embodiments, two or more time-subseries 94 may be inputted into an activity complexity dataset determination module 410 (e.g., such as activity complexity dataset determination module 120 described above with respect to FIG. 1).

Activity complexity dataset determination module 410 may determine an activity complexity dataset 420 based on two or more time-subseries 94. For example, if each of two or time-subseries 94 is processed to provide a 1D complexity data values probability distribution (e.g., as described above with respect to FIG. 3), activity complexity dataset 420 may include two- or more-dimensional activity complexity data values probability distribution.

For example, FIG. 4 depicts an example in which first time-subseries 94a, second time-subseries 94b, and third time-subseries 94c are processed to provide a first 1D complexity data values probability distribution 420a, a second 1D complexity data values probability distribution 420b and a third 1D complexity data values probability distribution 420c, respectively. First 1D complexity data values probability distribution 420a, second 1D complexity data values probability distribution 420b and third 1D complexity data values probability distribution 420c may be combined to provide activity complexity dataset 420 including 3D activity complexity data values probability distribution.

The pre-processing as described above with respect to FIG. 4 may be performed for any type of time series. For example, a time series may be divided into two time-subseries using a low pass and high pass filters. In another example, a time series may be analyzed using wavelet analysis to generate multiple time-subseries at different scales. In another example, one activity may be monitored by different sensors resulting in multiple time-subseries for each activity (e.g., as described above with respect to FIG. 1).

Reference is now made to FIG. 5, which is a schematic illustration of a second example of pre-pre-processing and of a process of determining an activity complexity dataset 520, according to some embodiments of the invention.

Some sensors may output multiple time series 95. For example, FIG. 5 depicts an example in which a sensor may output three time series 95. One example of such sensor may include an accelerometer. In this example, a first time series 95a may represent acceleration measured in an arbitrary X axis, a second time series 95b may represent acceleration measured in an arbitrary Y axis, and a third time series 95c may represent acceleration measured in an arbitrary Z axis.

Multiple time series 95 may be inputted into a pre-processing module 505 (e.g., such as pre-processing module 110 described above with respect to FIG. 1). In some embodiments, pre-processing module 505 may pre-process multiple time series 95 using dimension reduction analysis (e.g., such as Principal Components Analysis, t-Distributed Stochastic Neighbor Embedding (t-SNE), etc.) to yield a single time series 96.

Single time series 96 may be inputted into an activity complexity dataset determination module 510 (e.g., such as activity complexity dataset determination module 120 described above with respect to FIG. 1) that may determine an activity complexity dataset 520 (e.g., as described above with respect to FIGS. 1, 2, 3 and 4).

Reference is now made to FIG. 6, which is a schematic illustration of a user complexity model 600 including a multi-dimensional user complexity data values distribution, according to some embodiments of the invention.

FIG. 6 depicts an example of a user complexity model 600. User complexity model 600 may be determined by combining different activity complexity datasets 610 into a multivariate/multidimensional user complexity data values distribution, wherein each dimension of user complexity model may represent one of activity complexity datasets 610.

For example, FIG. 6 depicts an example of user complexity model 600 including a 3D user complexity data values distribution. A first dimension 602 of user complexity model 600 may represent a first activity complexity dataset 610a, a second dimension 604 of user complexity model 600 may represent a second activity complexity dataset 610b, and a third dimension 606 of user complexity model 600 may represent a third activity complexity dataset 610c.

It is noted that user complexity model 600 may in general include N-dimensional user complexity data values distribution (wherein N≥1). For example, if three activities are being monitored and an activity complexity dataset determined for each of the three activities includes a 3D activity complexity data values probability distribution (e.g., such as activity complexity dataset described above with respect to FIG. 4), a user complexity model may therefore include 9-dimensional activity complexity data values probability distribution.

Reference is now made to FIG. 7, which is a schematic illustration of a user complexity model 710, a reference user complexity model 720 and of a distance vector 730 therebetween, according to some embodiments of the invention.

FIG. 7 depicts a user complexity model 710 and a reference user complexity model 720. For example, user complexity model 710 and reference user complexity model 720 may be similar to the user complexity model and the reference user complexity model, respectively, described above with respect to FIG. 1.

A comparison between user complexity model 710 and reference user complexity mode 720 may be performed by, for example, a user complexity model determination module (e.g., such as user complexity model determination module 130 as described above with respect to FIG. 7). It is noted that FIG. 7 depicts user complexity model 710 and reference user complexity model 720 that include 3D complexity data values distribution for sake of clarity only and that complexity model 710 and reference user complexity model 720 may include complexity data values distribution of any dimension (e.g., as described above with respect to FIGS. 1 and 6).

FIG. 7 also depicts a distance vector 730 between user complexity model 710 and reference user complexity model 720. For example, distance vector 730 may be similar to the distance vector described above with respect to FIG. 1. Distance vector 730 may be determined by, for example, a distance vector determination module (e.g., such as distance vector determination module 160 as described above with respect to FIG. 1).

The number of dimensions of distance vector 730 may correspond to, for example, the number of dimensions of user complexity model 710. In example, depicted in FIG. 7, distance vector 730 may have 3D dimensions.

Reference is now made to FIG. 8, which is a schematic illustration of a reference anomaly probability logistic model 800, according to some embodiments of the invention.

FIG. 8 depicts an example of a reference anomaly probability logistic model 800. For example, reference anomaly probability logistic model 800 may be similar to the reference anomaly probability logistic model described above with respect to FIG. 1.

In some embodiments, reference anomaly probability logistic model 800 may be used, together with a distance vector between a user complexity model and a reference user complexity model, to determine a probability that a user has an anomaly (e.g., as described above with respect to FIG. 1). The reference anomaly probability logistic model may be determined based on, for example, training data collected from users who have been diagnosed with some level of, for example, health, cognitive and/or behavioral decline or with a deterioration of health, cognitive and/or behavioral condition that is known to affect cognitive ability.

FIG. 8 depicts an example of reference anomaly probability logistic model 800 derived for a norm of the distance vector (e.g., indicated in FIG. 8 as ∥\vec{D}∥). It is noted that reference anomaly probability logistic model 800 may be also derived for on one or more distance values in one or more dimensions of the distance vector (e.g., as described above).

Reference is now made to FIG. 9, which is a schematic illustration of a reference distance-condition model 900, according to some embodiments of the invention.

FIG. 9 depicts an example of a reference distance-condition model 900. For example, reference distance-condition model 900 may be similar to the reference distance-condition model described above with respect to FIG. 1. Reference distance-condition model 900 may be used, together with a distance vector between a user complexity model and a reference user complexity model, to determine a condition of a user (e.g., as described above with respect to FIG. 1).

FIG. 9 depicts an example of reference distance-condition model 900 derived for different dimensions of the distance vector (e.g., dimensions 912, 914, 916 as in example depicted in FIG. 9) and classified into groups of conditions (e.g., a first group 922 of conditions and a second group 024 of conditions). It is noted that reference distance-condition model 900 may be also derived for norm of the distance vector (e.g., as described above with respect to FIG. 1). It is also noted that reference distance-condition model 900 may be also derived for specific conditions rather than to groups 920 thereof (e.g., as described above with respect to FIG. 1).

Reference is now made to FIG. 10, which is a schematic illustration of a trend 1000 of distance vectors, according to some embodiments of the invention.

FIG. 10 depicts a trend 1000 of distance vectors as the time progresses, the distance vectors have been determined for different user complexity models determined for the same user during different monitoring time periods. Trend 1000 may be determined by, for example, a user condition tracking module (e.g., such as user condition tracking module 190 as described above with respect to FIG. 1).

FIG. 10 depicts an example of trend 1000 that is derived for distance values in different dimensions (e.g., dimensions 1010, 1012, 1014 as shown in FIG. 10) of the distance vectors. It is noted that trend 1000 may be also derived for norms of the distance vectors.

Trend 1000 of the distance vectors may be indicative of, for example, a trend of change in the condition of the user. For example, at time to, the user may not show any sign associated with health, cognitive and/or behavioral decline, but, as the time progresses, a distance (e.g., indicated in FIG. 10 by black rectangles “▪”) between the user complexity model and the reference user complexity model may, for example, increase, which may, for example, indicate the health, cognitive and/or behavioral decline thereof. Tracking and determining the trend of the distance vectors may, for example, enable an early detection of the condition of user 80 or a change thereof.

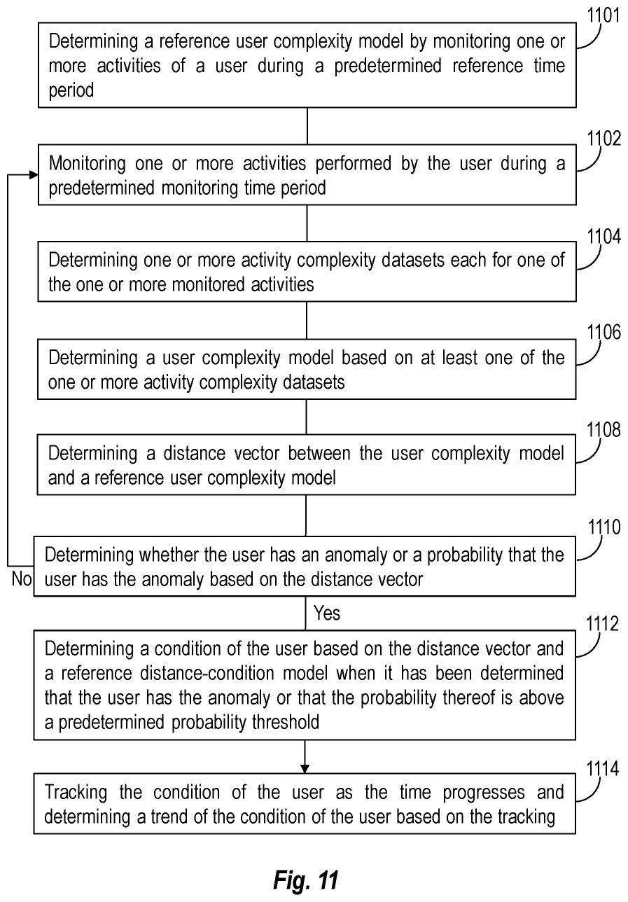

Reference is now made to FIG. 11, which is a flowchart of a method of determining a condition of a user, according to some embodiments of the invention.

The method may be implemented by system 100, which may be configured to implement the method. It is noted that the method is not limited to the flowcharts illustrated in FIG. 11 and to the corresponding description. For example, in various embodiments, the method need not move through each illustrated box or stage, or in exactly the same order as illustrated and described.

Some embodiments may include determining a reference user complexity model by monitoring one or more activities of a user during a predetermined reference time period (step 1101). The reference user complexity model may be determined by reference user complexity mode determination module 150 as described above with respect to FIG. 1. The reference user complexity model may be similar to reference user complexity models described above with respect to FIGS. 1 and 7.

Some embodiments may include monitoring one or more activities performed by the user during a predetermined monitoring time period (step 1102). The one or more activities may be monitored for 1-2 weeks. For example, the monitoring may include obtaining one or more time series of data points for each of the one or more monitored activities during a predetermined monitoring time period (e.g., as described above with respect to FIG. 1).

Some embodiments may include determining one or more activity complexity datasets each for one of the one or more monitored activities (step 1104). The one or more activity complexity datasets may be determined by, for example, activity complexity dataset determination module 120 of system 100 as described above with respect to FIG. 1. The one or more activity complexity datasets may be similar to, for example, activity complexity datasets described above with respect to FIGS. 1, 2, 3, 4 and 5. The one or more activity complexity datasets, each for one of the one or more monitored activities, may be determined based on, for example, at least one of the one or more time series obtained for the respective monitored activity (e.g., as described above with respect to FIG. 1).

Some embodiments may include determining a user complexity model based on at least one of the one or more activity complexity datasets (step 1106). The user complexity model may be determined by, for example, user complexity model determination module 130 as described above with respect to FIG. 1. The user complexity model may be similar to, for example, user complexity models described above with respect to FIGS. 1, 6 and 7. The user complexity model may be determined by, for example, combining different activity complexity datasets determined for different monitored activities (e.g., as described above with respect to FIGS. 1 and 6).

Some embodiments may include determining a distance vector between the user complexity model and the reference user complexity model (step 1108). The distance vector may be determined by, for example, distance vector determination module 160 as described above with respect to FIG. 1. The distance vector and reference user complexity model may be similar to, for example, distance vectors and reference user complexity models, respectively, described above with respect to FIGS. 1 and 7.

Various embodiments may include determining whether the user has an anomaly or a probability that the user has the anomaly based on the distance vector (step 1110). For example, as described above with respect to FIGS. 1 and 8. For example, one or more predetermined thresholds, a reference anomaly probability logistic model and/or trained machine learning method may be applied to the distance vector or to one or more distance values in one or more dimensions of the distance vector to determine whether the user has the anomaly or a probability thereof.

Some embodiments may include repeating step 1102 when it has been determined that the user has no anomaly.

Various embodiments may include determining a condition of the user based on the distance vector and a reference distance-condition model when it has been determined that the user has the anomaly or that the probability thereof is above a predetermined probability threshold (step 1112). For example, the condition of the user may be determined by user condition determination module 180 as described above with respect to FIG. 1. The reference distance-condition model may be similar to, for example, reference distance-condition models described above with respect to FIGS. 1 and 9. The condition of the user may, for example, be health, cognitive and/or behavioral condition. In various embodiments, the condition of the user may relate to a specific health, cognitive and/or behavior condition or to a group of health, cognitive and/or behavior conditions.

Some embodiments may include tracking the condition of the user as the time progresses and determining a trend of the condition of the user based on the tracking thereof (step 1114). For example, the tracking of the condition and determining of the trend thereof in time may be performed user condition tracking module 190 as described above with respect to FIGS. 1 and 10.

Advantageously, the disclosed system and method may enable determining condition-specific health, cognitive and/or behavior anomalies and the trends thereof for a user and/or recovery of the user to a normal condition based on monitored daily activity of the user.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions thereof. The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram portion or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams can represent a module, segment, or portion of code, which includes one or more executable instructions for implementing the specified logical function(s). It should also be noted that, in some alternative implementations, the functions noted in the portion can occur out of the order noted in the figures. For example, two portions shown in succession can, in fact, be executed substantially concurrently, or the portions can sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method of determining a condition of a user, the method comprising:
monitoring one or more activities performed by a user during a predetermined monitoring time period;
determining one or more activity complexity datasets each for one of the one or more monitored activities;
determining a user complexity model based on at least one of the one or more activity complexity datasets;
determining a distance vector between the user complexity model and a reference user complexity model; and
determining a condition of the user based on the distance vector and a reference distance-condition model.

2. The method of claim 1, further comprising:
monitoring the one or more activities by obtaining one or more time series of data points for each of the one or more monitored activities during the predetermined monitoring time period from one or more sensors wearable by the user; and
determining the one or more activity complexity datasets, each for one of the one or more monitored activities, based on at least one of the one or more time series obtained for the respective monitored activity.

3. The method of claim 1, further comprising:
determining at least one of: whether the user has an anomaly and a probability that the user has the anomaly, based on the distance vector; and
determining the condition of the user when it has been determined that the user has the anomaly or when the probability thereof is above a predetermined probability threshold.

4. The method of claim 3, further comprising determining whether the user has the anomaly based on at least one of:
a norm of the distance vector and a predetermined norm threshold;
one or more distance values in one or more dimensions of the distance vector and one or more predetermined distance thresholds; and
a pre-trained machine learning model.

5. The method of claim 3, further comprising determining the probability that the user has the anomaly based on at least one of:
a reference anomaly probability logistic model; and
a pre-trained machine learning model.

6. The method of claim 1, further comprising determining a condition of the user based on at least one of:
a norm of the distance vector; and
one or more distance values in one or more dimensions of the distance vector.

7. The method of claim 1, wherein determining the condition of the user comprises determining one of:
a specific condition of the user; or
a group of conditions from which the user may suffer.

8. The method of claim 1, further comprising:
tracking the condition of the user as the time progresses; and
determining a trend of the condition of the user based on the tracking thereof.

9. The method of claim 1, further comprising predetermining the reference user complexity model by monitoring the one or more activities during a predetermined reference time period prior to the predetermined monitoring time period.

10. A system for determining a condition of a user, the system comprising:
an activity complexity dataset determination module configured to:
obtain one or more time series of data points for each of one or more monitored activities during a predetermined monitoring time period, and
determine the one or more activity complexity datasets, each for one of the one or more monitored activities, based on at least one of the one or more time series obtained for the respective monitored activity;
a user complexity model determination module configured to determine a user complexity model based on at least one of the one or more activity complexity datasets;
a distance vector determination module configured to determine a distance vector between the user complexity model and a reference user complexity model; and
a user condition determination module configured to determine a condition of the user based on the distance vector and a reference distance-condition model.

11. The system of claim 10, further comprising:
an anomaly determination model that is configured to determine at least one of: whether the user has an anomaly and a probability that the user has the anomaly, based on the distance vector; and
wherein the user condition determination model is configured to determine the condition of the user when it has been determined that the user has the anomaly or when the probability thereof is above a predetermined probability threshold.

12. The system of claim 11, wherein the anomaly determination model is further configured to determine whether the user has the anomaly based on at least one of:
a norm of the distance vector and a predetermined norm threshold;
one or more distance values in one or more dimensions of the distance vector and one or more predetermined distance thresholds; and
a pre-trained machine learning model.

13. The system of claim 11, wherein the anomaly determination model is further configured to determine the probability that the user has the anomaly based on at least one of:
a reference anomaly probability logistic model; and
a pre-trained machine learning model.

14. The system of claim 10, wherein the user condition determination module is configured to determine a condition of the user based on at least one of:
a norm of the distance vector; and
one or more distance values in one or more dimensions of the distance vector.

15. The system of claim 10, wherein the user condition determination module is configured to determine one of:
a specific condition of the user; or
a group of conditions from which the user may suffer.

16. The system of claim 10, further comprising a user condition tracking module configured to:
track the condition of the user as the time progresses; and
determine a trend of the condition of the user based on the tracking thereof.

17. The system of claim 10, further comprising a reference user complexity model determination module configured to predetermine the reference user complexity model by monitoring the one or more activities during a predetermined reference time period prior to the predetermined monitoring time period.
